(19) United States
(12) Patent Application Publication
(10) Pub. No.: US 2024/0325646 A1
(43) Pub. Date: Oct. 3, 2024

(54) INJECTOR NEEDLE CAP AND/OR LINER REMOVER

(71) Applicant: West Pharma. Services IL, Ltd., Ra'anana (IL)

(72) Inventors: Oz CABIRI, Macabim-Reut (IL); Ran HEZKIAHU, Herzliya (IL)

(21) Appl. No.: 18/737,954

(22) Filed: Jun. 8, 2024

Related U.S. Application Data
(62) Division of application No. 17/696,521, filed on Mar. 16, 2022, now Pat. No. 12,036,394.
Application No. 17/696,521 is a division of application No. 15/766,670, filed on Apr. 6, 2018, now Pat. No. 11,318,254.
Application No. 15/766,670 is a continuation of application No. 15/204,542, filed on Jul. 7, 2016, now Pat. No. 10,576,207.
Application No. PCT/US16/56247 is a continuation of application No. 15/269,248, filed on Sep. 19, 2016, now Pat. No. 10,086,145.
(60) Provisional application No. 62/281,536, filed on Jan. 21, 2016.
(60) Provisional application No. 62/284,806, filed on Oct. 9, 2015.

(51) Int. Cl. A61M 5/28; A61M 5/142; A61M 5/145; A61M 5/158; A61M 5/31; A61M 5/32; A61M 5/34; B29C 33/12; B29L 31/00; B65D 1/36; B65D 5/50; B65D 21/02; B65D 25/10

(52) U.S. Cl. CPC A61M 5/28; A61M 5/3134; A61M 5/3202; A61M 5/3204; A61M 5/34; B29C 33/12; B65D 1/36; B65D 5/503; B65D 21/0233; B65D 25/108; A61M 5/14248; A61M 5/1456; A61M 2005/1581; A61M 2005/312; A61M 2005/341; A61M 2205/3306; A61M 2207/00; B29L 2031/7544

(57) ABSTRACT

A system for medicament delivery includes a cartridge containing a medicament and in fluid communication with a needle, where the needle is enveloped by a needle cap, a housing for containing the cartridge and having an orifice for allowing access to said needle cap and its associated needle, and a needle cap remover device.

### CROSS-REFERENCE TO RELATED APPLICATION

This application is a divisional application of U.S. patent application Ser. No. 17/696,521, filed Mar. 16, 2022, which is a divisional application of U.S. patent application Ser. No. 15/766,670, filed Apr. 6, 2018, and issued as U.S. Pat. No. 11,318,254 on May 3, 2022, which is a U.S. National Phase Patent Application of International Application No. PCT/US16/56247, filed Oct. 10, 2016, which is a continuation of U.S. patent application Ser. No. 15/204,542, filed Jul. 7, 2016, and issued as U.S. Pat. No. 10,576,207 on Mar. 3, 2020, which claims the benefit of U.S. Provisional Patent Application No. 62/281,536, filed Jan. 21, 2016, and U.S. Provisional Patent Application No. 62/284,806, filed Oct. 9, 2015; and International Application No. PCT/US16/56247 is a continuation of U.S. patent application Ser. No. 15/269,248, filed Sep. 19, 2016, and issued as U.S. Pat. No. 10,086,145 on Oct. 2, 2018, the disclosures of which are incorporated herein by reference.

### FIELD AND BACKGROUND OF THE INVENTION

The present invention, in some embodiments thereof, relates to a needle cap remover and, more particularly, but not exclusively, to a bifurcated needle cap remover.

U.S. Patent Application No. US 2012/0238961 discloses “a needle shield remover that reliably engages with a distal cap of an automatic injection device and with one or more needle shields coupled to a syringe of the device. When a user removes the distal cap, the needle shield remover reliably removes the needle shields (e.g., a soft needle shield and a rigid needle shield) from the syringe, thereby exposing the injection needle for performing an injection. In an exemplary assembly method, a needle shield remover is engaged to a needle shield coupled to a syringe, prior to insertion of the syringe and needle shield remover assembly into a housing of the device. This exemplary assembly method allows visual inspection, outside the housing of the device, to ensure that the needle shield remover is correctly and reliably engaged to the needle shield before the syringe and needle shield remover assembly is inserted into the housing”.

U.S. Pat. No. 6,843,782 discloses “a drug delivery device having a base member defining a skin-contacting surface, a syringe serving as a reservoir for the drug, and means for expelling drug from the syringe. The syringe is connected to the base member such that the longitudinal axis of the syringe is substantially parallel to the skin surface. A delivery needle is in communication with the syringe. The needle has an angled bend which directs the tip of the needle substantially perpendicular to the skin-contacting surface. In use, the tip of the needle is adapted to penetrate the skin of the subject”.

International Patent Application Publication No. WO 2015/048791 discloses “a method of preparing a compound device for use. The device may include a sealed component and an active outer surface. The outer surface may be protected by a surface cover. Preparing the device may include activating the active outer surface by removing the surface cover and exposing an internal portion of the sealed component to the exterior of the device by unsealing the sealed component and synchronizing the activating and said unsealing using a coupler attached to the surface cover and the sealed component.”

### SUMMARY OF THE INVENTION

Example 1. A device for removing a needle cap, the needle cap shielding a needle of an injector system, the device comprising: an elongated hollow body, having a distal end sized and shaped to at least partially envelop the needle cap; and at least one snap coupled to at least one respective element of the elongated body; the elongated body having a closed and open configuration, wherein the closed configuration is defined by the at least one snap defining a width smaller than a width of a top sill of the needle cap, and the open configuration is defined by the at least one snap having a width wide enough to fit at least the width of the top sill; wherein the hollow body in the closed configuration is shaped to hold the at least one snap overhanging a top sill of the needle cap with the hollow body enveloping a lower portion of the needle cap.

Example 2. The device of example 1, wherein the at least one snap inflects towards a central axis of the needle cap, being angled with respect to the axis.

Example 3. The device of any of examples 1-2, comprising at least two snaps coupled to at least two respective elements being at least two elastic arms having an open configuration defined by elastically deflecting away from the central axis of the needle cap, and wherein once the at least two snaps are pushed beyond the top sill of the needle cap, the elastic arms return to their closed configuration and the at least two snaps overhang the top sill of the needle cap.

Example 4. The device of example 3, wherein each of the at least two elastic arms is defined as a surface between two slits, the two slits extend from the distal end of the elongated hollow body.

Example 5. The device of example 4, wherein the slits extend to a length having a range of between about 20% and about 60% of a length of the elongated hollow body.

Example 6. The device of any of examples 4-5, wherein each of the two slits have a width having a range of about 0.5 mm and about 1.5 mm.

Example 7. The device of any of examples 4-6, further comprising at least one intermediate longitudinal slit provided in the surface between the two slits.

Example 8. The device of any of examples 3-7, wherein the at least two arms encompass no more than about 40% of a circumference of the elongated hollow body.

Example 9. The device of example 8, wherein the at least two arms are symmetrically positioned around the circumference of the elongated hollow body.

Example 10. The device of any of examples 1-9, wherein the elongated body is configured to be pushed over a needle cap using a force having a range of about 50 g and about 200 g.

Example 11. The device of any of examples 1-10, wherein the elongated body further comprises at least two guides along its inner surface and oriented along a longitudinal axis of the elongated body, the at least two guides sized and shaped to accommodate complementary elements positioned on an outer surface of the needle cap.

Example 12. The device of any of examples 1-11, further comprising a connector sized and shaped to fit a proximal portion of the body, the connector comprising a user handle having a protruding element.

Example 13. The device of example 12, wherein the elongated body further comprises at least one interlocking element complementary to an interlocking member positioned in the connector, inhibiting a lateral movement of the elongated body with respect to the connector.

Example 14. A system for medicament delivery, comprising: a cartridge containing a medicament and being in fluid communication with a needle, the needle enveloped by a needle cap; a housing for containing the cartridge and having an orifice for allowing access to the needle cap and its associated needle; and a needle cap remover device comprising: at least two elastic arms coupled to define an unstressed width greater than the outside of a width of the top sill of the needle cap, the elastic arms envelop at least a portion of the needle cap, wherein a proximal end of the elastic arms can be accessed through the orifice; and at least two snaps, each coupled to a distal end of each of the at least two elastic arms, the snaps defining an unstressed width smaller than the width of the top sill of the needle cap; wherein the at least two snaps overhang the top sill of the needle cap, and when the proximal end of the elastic arms is pulled, the snaps cause the needle cap to pull with the needle cap remover device.

Example 15. The system of example 14, wherein each of the at least two snaps comprise two hooks inflecting towards a central axis of the needle cap and being angled towards the top surface of the needle cap.

Example 16. The system of example 15, wherein the hooks are symmetrically arranged around a central axis of the top sill of the needle cap.

Example 17. The system of any of examples 15-16, wherein the needle cap remover device comprises two of the at least two arms, and wherein each of the two arms comprises two of the hooks.

Example 18. The system of any of examples 14-17, further comprising an adhesive layer having a protective liner, the adhesive layer connected to the housing, wherein the protective liner extends beyond a surface of the housing and positioned between the cap remover body and a connector.

Example 19. The system of example 18, wherein when the needle cap remover is pulled linearly the protective liner is peeled from the housing.

Example 20. The system of any of examples 14-19, wherein pulling the cap remover body together with the enveloped needle cap requires a force being no more than 1 kg at most.

Example 21. A method of assembling a needle cap remover onto a needle cap, comprising: aligning a longitudinal axis of the needle cap remover body to be colinear with a longitudinal axis of the needle cap; inserting the distal end of the cap remover body in the direction of the top portion of the needle cap while deflecting at least two elastic elements of the cap remover body in a direction away from the central axis of the needle cap, the deflecting is provided by a plurality of hooks coupled to a distal end of the elastic members and defining a smaller perimeter than a perimeter of the needle cap; wherein the inserting is provided until the plurality of hooks extend beyond the top portion of the needle cap and snap inwardly towards the central axis of the needle cap, thereby overhanging the top portion of the needle cap, causing the elastic elements to snap from the deflecting towards the central axis of the needle cap.

Example 22. The method according to example 21, wherein the needle cap is comprised within an injector system and the method further comprises inserting the needle cap remover through an orifice in the injector system having access to the needle cap.

Example 23. The method according to any of examples 21-22, further comprising interlocking a proximal portion of the needle cap remover body with a connector having a user handle.

Example 24. The method of example 16, further comprising associating the cap remover body with a battery insulator.

Example 25. A cover for an injector having a needle, a needle cap, and a needle cap remover having a coupler for a needle cap and a user handle, the user handle having a protruding element opposite the coupler, the protruding element having a short dimension and a long dimension, the cover comprising: at least one adhesive layer covering a surface of an outside of the injector; at least one liner layer, covering the adhesive layer, an extension extending beyond the surface; a first aperture through the adhesive layer and the liner layer aligned to an opening in the surface, the first aperture sized and shaped for passage of the coupler therethrough; and a second elongated aperture, through the extension, having a length being longer than the short dimension and shorter than the long dimension of the protruding element.

Example 26. The cover according to example 25, wherein the extension and the liner are made of a single integral piece of material

Example 27. The cover according to example 25, wherein the second elongated aperture has a width being smaller than a shoulder portion extending from the protruding element along its long dimension.

Example 28. The cover according to example 27, wherein the extension comprises a resilient material enabling enough stretch to pass over the shoulder and to overhang a top sill of the shoulder.

Example 29. The cover according to example 28, wherein the resilient material residing in at least a portion of a circumference of the second elongated aperture.

Example 30. The cover according to any of examples 25-29, further comprising a rigid layer surrounding at least a portion of a circumference of the second elongated aperture.

Example 31. The cover according to any of examples 25-30, wherein the first aperture having a circumference shape matching a circumference shape of the needle cap remover.

Example 32. The cover according to example 31, wherein a width of the circumference shape of the first aperture is wider than the circumference shape of the needle cap remover by no more than about 1 mm.

Example 33. The cover according to any of examples 25-32, further comprising at least one intermittent supporting layer attached to the at least one liner layer, the intermittent supporting layer is not continuous over an area of the cover;

Example 34. A method of assembling an automatic injector device having an interior volume and a base located between the interior volume and an exterior surface defined by the base, the base having an opening, the method comprising: supplying a cover having a first aperture aligned with the opening in the base, the cover having an extension extending beyond the exterior surface of the base and having a second aperture; passing a distal portion of a needle cap remover through the first aperture and the opening; coupling the distal portion to a needle cap positioned in the interior volume; bending the extension over a protruding element provided in a proximal end of the needle cap remover; the protruding element having a long dimension and a short dimension; orienting the second aperture of the cover to face the short dimension of the handle and rotating the cover onto the protruding element until the second aperture is oriented to face the long dimension.

Example 35. The method of example 34, further comprising: interlocking a cartridge having a capped needle in the inner portion of the base, the cartridge central axis perpendicular to the cover and the capped needle centrally aligned with the first aperture of the cover and the opening of the base.

Example 36. The method of any of examples 34-35, wherein the cover converts a linear movement away from the exterior surface into a peeling force on the cover.

Example 37. The method of example any of examples 34-36, further comprising sliding the second aperture along the long dimension in an offset direction from a central axis of the long dimension.

Example 38. The method of any of examples 34-37, further comprising stabilizing the extension of the cover onto the protruding element by constraining the second aperture against a widening portion of the protruding element.

Example 39. The method of example 38, wherein the constraining further includes passing the second aperture over the widening portion into a narrower portion and overhanging an edge of the second aperture over the widening portion.

Example 40. The method of any of examples 34-39, further comprising rotating the user handle such that the long dimension is not aligned with the length of the second aperture.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E schematically illustrate an exemplary device for removing a needle cap having a bifurcated body, in accordance with some embodiments of the current invention, wherein FIG. 1A illustrates a perspective view, FIG. 1B illustrates a front view, FIG. 1C illustrates a side view, FIG. 1D illustrates a top view and FIG. 1E illustrates a perspective top view;

FIGS. 2A-C schematically illustrate an exemplary use of the cap remover in an injector device, in accordance with some embodiments of the current invention, wherein FIG. 2A illustrates an example of the device being used in an automatic injector assembly, FIG. 2B illustrates the inner cartridge and needle assembly provided in the injector of FIG. 2A, and FIG. 2C illustrates another example of the device used in an automatic injector;

FIGS. 3A-G schematically illustrate an exemplary incorporation of an adhesive liner of an injector device into the cap remover, in accordance with some embodiments of the current invention, wherein FIG. 3A illustrates a perspective side view, FIG. 3B illustrates a side view, FIG. 3C illustrates a cross sectional side view of a cap remover assembled onto a cartridge, FIG. 3D illustrates a perspective view of the injector having a device cover, FIG. 3E illustrates a front view of the injector having a device cover, FIG. 3F illustrates a perspective view of the device cover and FIG. 3G illustrates an explosive view of the device cover layers, in accordance with some embodiments of the invention;

FIGS. 4A-I schematically illustrate an exemplary needle cap remover bifurcated cover and its assembly with a needle cap, in accordance with some embodiments of the current invention, wherein FIG. 4A illustrates a perspective view, FIG. 4B illustrates a front view, FIG. 4C illustrates a side view, FIG. 4D illustrates a top view, FIG. 4E illustrates a bottom view of a needle cap remover embodiment, and FIG. 4E illustrates a second needle cap remover cover embodiment, shown as part of a cap remover device assembled on a cartridge in FIG. 4F, and FIG. 4G illustrates a cross section of a front view of a needle cap remover device being pushed onto a needle cap and FIG. 4H illustrates a cross section of the device and the needle cap after their assembly, and FIG. 4I illustrates a partial perspective close up view of the top portion of the needle cap assembled with the cap remover;

FIGS. 5A-E schematically illustrate an exemplary cap remover connector having a handle, in accordance with some embodiments of the current invention, wherein FIG. 5A illustrates a perspective view, FIG. 5B illustrates a cross section view, FIG. 5C illustrates a top view, FIG. 5D illustrates a front view and FIG. 5E illustrates a side view;

FIG. 6 is a flow chart illustrating an exemplary process for assembling a needle cap remover onto a needle cap, in accordance with some embodiments of the current invention;

FIG. 7 is a flow chart illustrating an exemplary assembly of a needle cap remover assembly with an adhesive liner, in accordance with some embodiments of the current invention;

FIG. 8 is a flow chart illustrating an exemplary process assembling an automatic injector device, in accordance with some embodiments of the current invention;

FIGS. 9A-B schematically illustrate an exemplary multi-part cap remover, in accordance with some embodiments of the current invention, wherein FIG. 9A exemplifies a multi-part cap remover in an open configuration and FIG. 9B exemplifies a multi-part cap remover in a tight configuration;

FIG. 10 is a flow chart illustrating a process of assembling a multi-part cap remover onto a needle cap, in accordance with some embodiments of the current invention; and

FIGS. 11A-C exemplify a singled arm needle cap remover, in accordance with some embodiments of the current invention, wherein FIG. 11A illustrates a perspective view of the device, FIG. 11B illustrates a cross-section view of the device and FIG. 11C illustrates a top view of the device.

### DESCRIPTION OF SPECIFIC EMBODIMENTS OF THE INVENTION

The present invention, in some embodiments thereof, relates to a needle cap remover and, more particularly, but not exclusively, to a bifurcated needle cap remover.

### Overview

An aspect of several embodiments of the invention relates to a needle cap remover configured to slide in one direction and clamp in the opposite direction. In some embodiments, the needle cap remover comprises a cover body having a longitudinal axis characterized by a distal end configured to be pushed over and/or receiving and/or enveloping a needle cap, and a proximal end configured for being pulled by a user. Optionally the body is cylindrical. Alternatively, the body includes a conical section. Alternatively or additionally, the body comprises at least two longitudinal arms at least partially coupled to define a tubular structure, optionally having a diameter larger than a diameter of a needle cap.

In some embodiments, the cover body comprises at least two hooks, optionally coupled to its distal end. In some embodiments, the hooks are provided at the distal portion of the longitudinal arms. Optionally, the hooks define a diameter which is smaller than the diameter of the needle cap. In some embodiments, once the cover body is pushed over the needle cap, the hooks are deflected away from the central axis of the needle cap to allow their defined diameter to fit over the larger diameter of the needle cap. In some embodiments, deflection of the hooks is provided by elastic elements, optionally the elastic elements comprise the at least two longitudinal arms.

In some embodiments, the at least two longitudinal arms are defined by bifurcated sections in the cover body. Optionally, the bifurcated sections are configured to distribute pushing forces, potentially enabling assembly of the needle cap remover onto the needle cap without exerting too much force onto the needle cap, for example exerting no more than 100 g force, or exerting no more than 150 g force, or exerting no more than 200 g force, or exerting no more than 250 g force. Alternatively or additionally, the bifurcated sections are configured to distribute pulling forces such that potentially removing the needle cap with the needle cap remover substantially maintains an axial direction. In some embodiments, in order to pull the needle cap no more than 0.5 Kg force is applied. Alternatively, no more than 0.7 Kg force is applied. Alternatively, no more than 0.9 Kg force is applied. Alternatively, no more than 1 Kg force is applied. Alternatively, no more than 1.2 Kg force is applied. Alternatively, no more than 1.5 Kg force is applied. In some embodiments, the cap remover cover body has an integral proximal end and a bifurcated distal end.

In some embodiments, the bifurcated end of the cap remover comprises 4 slits which divide the bifurcated end into 4 longitudinal arms. Alternatively or additionally, the bifurcated end of the cap remover comprises 5, or 6, or 7, or 8 slits. Alternatively or additionally, the bifurcated end of the cap remover comprises 2, or 3 slits. Optionally, the slits are offset with respect to the longitudinal axis of the cover body, for example, tilted by an angle range of 1°-20°. Alternatively, at least some of the slits are tilted by an angle range of 15°-35°. Alternatively at least some of the slits are tilted by an angle range of 30°-45°, or any range smaller, larger or intermediate. Optionally, the slits define a triangular portion.

In some embodiments, the bifurcations are arranged symmetrically around a perimeter of the cap remover. Optionally, the bifurcations are arranged equidistantly. Alternatively, the bifurcations are arranged such that at least two different sizes of bifurcated portions, optionally in the form of elongated arms, are provided. Optionally, pairs of equally sized bifurcated portions are arranged substantially diametrically.

In some embodiments, bifurcation portions are geometrically distributed in a configuration requiring a relatively small force when pushing the cap remover onto a needle cap and a relatively large force when pulling the cap with the cap remover. Potentially, the relative location of the slits affects the directionality of the forces which are exerted on the cap remover. Alternatively or additionally, the elasticity of at least a portion of the bifurcated portions affects the directionality of the forces exerted on the cover body. In some embodiments, elasticity of the cap remover portions is affected by the perimeter length taken up by the portions. Alternatively or additionally, elasticity of the cap remover portions is affected by the composition of the portions material.

In some embodiments, the distal portion of the longitudinal arms is coupled to at least two hooks, and/or fingers. Optionally, the hooks cause the perimeter of the cap remover to be smaller than the perimeter of the needle cap. In some embodiments, when the cover body is pushed over the needle cap, once the hooks pass the top sill of the needle cap, the smaller perimeter allows the hooks to overhang the top sill. In some embodiments, at least some of the bifurcated portions, optionally in the form of longitudinal arms, are elastic enough to deflect away from the longitudinal axis of the cap remover at least to an extent which allows the smaller perimeter defined by the hooks to fit over the larger perimeter of the needle cap.

Optionally, the hooks operate at a snap-fit mechanism. In some embodiments, at least some of the hooks include projections extending towards a central axis of the cap remover. In some embodiments, when the cap remover is pushed over the needle cap, once the hooks pass over the top sill of the needle cap, the hooks and/or elastic arms bounce back and/or snap towards the central longitudinal axis. In some embodiments, when a pulling force is applied to a cover body assembled onto a needle cap, the hooks are configured to exert force in an axial direction towards to the bottom portion of the needle cap. Optionally, hooks in the form of inward projections include projections which are tilted towards the bottom portion of the needle cap, potentially creating a resisting clasp when the cover body is pulled and the projections are pushed against the top sill of the needle cap. Alternatively or additionally, snap projections are perpendicular to the longitudinal axis of the cover body.

In some embodiments, the position of the slits affects the resistance exerted by the snaps which are being pushed on to the needle cap, optionally leading to the pulling of the needle cap with the cap remover when pulling the cap remover. Alternatively or additionally, the size of the slits affects the snaps being held. In some embodiments, the position configuration of the slits allow the clastic portions to deflect away from the central axis of the cap remover when pushed over the needle cap in one direction, but at the same time cause the snaps to hold onto the needle cap when the cap remover is pulled in the opposite direction, and prevent the clastic portions from deflecting. In some embodiments, intermediate slits are provided, optionally in the clastic portions, optionally positioned in proximity to the middle portion of the longitudinal axis of the cap remover. In some embodiments, intermediate slits do not reach the edge of the cap remover body.

In some embodiments, the bifurcated portions are optionally symmetrically arranged around the perimeter of the cap remover. Alternatively or additionally, the bifurcated portions are symmetrically arranged with respect to a central axis. In some embodiments, four slits are provided, partitioning the cap remover body distal end into four portions, optionally two clastic portions and two rigid portions. Elastic portions are for example arms which can deflect away from the central axis of the cover body to at least allow coupled hooks to be fit over the perimeter of the needle cap. Rigid portions are for example portions of the cover body which are not configured to deflect away from a central axis of the cover body. In some embodiments, elastic portions are positioned substantially diametrically. Alternatively or additionally, rigid portions are positioned substantially diametrically.

In some embodiments, the elastic portions take up a smaller perimeter length with respect to the rigid portions. For example, the clastic portions comprise 10-40% of the body cover perimeter, while the rigid portions comprise 90-60% of the body cover perimeter. Alternatively, the elastic portions comprise 20-30% of the body cover perimeter, while the rigid portions comprise 80-70% of the body cover perimeter. It is a potential advantage to provide larger rigid portions and smaller elastic portions, as it contributes to the rigid and clastic characteristics of the portions, respectively.

In some embodiments, a connector, for example a body containing a clamping mechanism, is provided to be assembled onto the cap remover cover body, optionally to connect the cover body to other features. Alternatively or additionally, the connector acts as a securing mechanism and is optionally shaped and sized to add mechanical stability to the cover body. In some embodiments, the cover body is positioned within an injector system and the connector can be operated from the outside of the injector system. In some embodiments, the cap remover comprises fasteners configured to interlock with the connector. Potentially, the fasteners prevent lateral movement of the cover body with respect to the needle cap. Optionally, the connector further comprises a user handle. In some embodiments, the fasteners interlocking with the connector limit force exertion in a rotational direction, potentially precluding sliding of the needle remover body around the needle cap and/or preserving an orientation of the needle cap remover with respect to the needle cap.

An aspect of several embodiments of the invention relates to a medicament delivery system, for example an autoinjector comprising a needle cap remover. In some embodiments, a needle is installed into an autoinjector and is covered with a needle cap. Subsequently, an adhesive liner is installed to the injector. In some embodiments, removal of the needle cap also brings about removal of the adhesive liner. In some embodiments, the needle cap remover is positioned perpendicularly to the base of the injector system, to allow a user a more intuitive pulling direction.

An aspect of several embodiments of the invention relates to a needle cap remover having at least two separate fastening mechanisms. In some embodiments, a first fastening mechanism comprises at least two enveloping arms sized and shaped to envelop at least a portion of a needle cap. In some embodiments, the arms are coupled in at least a portion across their longitudinal axis, optionally enabling the arms to hinge-tilt from the coupled position. Optionally, when the distal portions of the arms tilted away from the longitudinal axis of the cap remover, the arms define an open configuration, and when the arms are not tilted away they define a closed enveloping configuration. In some embodiments, a second fastening mechanism is configured to latch the arms in their closed configuration, optionally in the form of a connector which may also connect arms being inside an injector system to an outside of the injector system.

In some embodiments, a closed configuration is defined by having the members enclosed around the needle cap. An opened configuration is defined by having the members spaced to have a diameter greater than the needle cap, optionally, at least as wide as the widest diameter of the needle cap. In some embodiments, the arms are provided with fingers that are configured to overhang a top sill of the needle cap once the arms are in their closed configurations. Optionally, when the cap remover is being pulled, the connector keeps the arms in their closed configuration and the fingers push down on the cap and remove it from the needle, optionally in substantially a downwards axial direction.

An aspect of some embodiments of the invention relates to a device protective cover allowing access of a needle cap remover into an inner portion of the device, and designed to be assembled onto the needle cap remover and its user pulling handle after the remover is assembled with its user handle. In some embodiments, the protective cover comprises a plurality of layers, optionally at least an adhesive layer and a liner layer protecting the adhesive surface of the adhesive layer. In some embodiments, protective cover is designed to be removed by pulling the needle cap remover through its user handle in a linear direction. Optionally, only a portion of the protective cover area is attached to the injector, while an extending edge extends from the end of the portion attached. In some embodiments, the extended edge is folded and/or bent to be oriented facing the attached portion, optionally by handing the extended edge onto the user handle.

In some embodiments, the protective cover comprises at least two apertures. Optionally, a first aperture allows insertion of the needle cap remover into the injector inner portion. In some embodiments, the first aperture is sized to match the circumferential shape of the needle cap remover, optionally being wider than the needle cap remover. It is a potential advantage to keep the first aperture small enough to allow sufficient stability when the device is adhered to the patient and the needle is penetrating through this aperture. It is another potential advantage to design the aperture to have a width smaller than a human finger, to prevent a user from inserting his figure into the needle region, e.g. smaller than 15 mm, and/or smaller than 10 mm, and/or smaller than 5 mm. In some embodiments, the cap remover body has no outward protrusions in the region which needs to be inserted into the aperture, in order to preserve the option for a small aperture width as allowed by the body's own width.

Also optionally, a second aperture allows fitting over the user handle. In some embodiments, a user handle is provided as having a long dimension and a short dimension. Optionally, the second aperture is sized as an elongated slit, having a length being longer than the long dimension and shorter than the short dimension of the handle. In some embodiments, the elongated aperture is assembled onto the handle by first orienting the aperture to face the shorter dimension and then rotating the edge of the cover such that the elongated aperture slides towards the longer dimension, optionally eventually being oriented to face the long dimension. In some embodiments, when placing the liner over the handle, no force is applied to the cap remover and/or needle cap. Alternatively, a force smaller than 50 g, and/or 100 g, and/or 150 g is applied.

In some embodiments, the edge is stabilized in place by the handle becoming wider as the aperture slides toward the long dimension and by constraining the edge of the aperture against the wide portion of the handle, being wider than the aperture. Alternatively, the edge is directed to pass the widest portion of the handle and reach a narrower portion, causing the aperture edge to overhang over the wide portion serving as a shoulder portion. Alternatively or additionally, the handle is rotated after the edge is passed over it, such that the long dimension of the handle is found at an angle to the length of the aperture, thereby preventing a slide of the edge in the direction it passed over. Alternatively or additionally, the edge slides to an offset direction from the center of the long dimension, misaligning the aperture with the handle's long dimension.

In some embodiments, at least a portion of an edge of at least one of the apertures also comprises a supporting layer in the form of a rigid layer, rigid enough to provide mechanical stability to prevent bending at the forces which are applied when pulling the cover. Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Embodiments

### 1 An Exemplary Needle Cap Remover

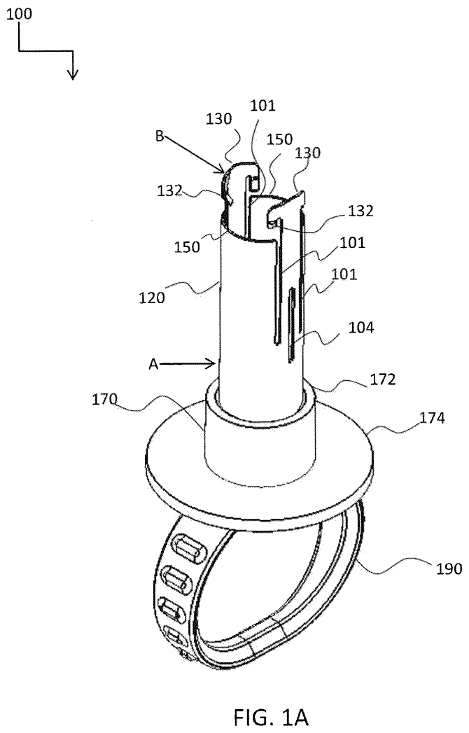

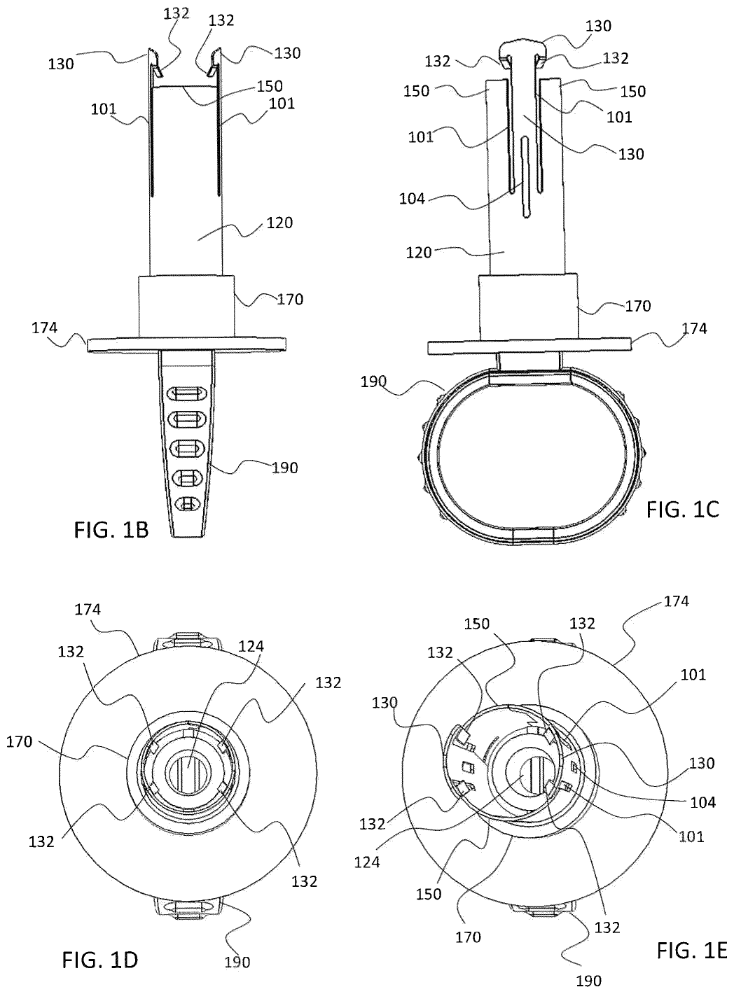

Referring now to the drawings, FIGS. 1A-E illustrate a needle cap remover 100 designed to remove a needle cover in accordance with an embodiment of the current invention. In some embodiments, needle cap remover 100 is designed such that forces exerted on the needle cover do not disturb a sterility state of a needle cap enveloping a sterile needle.

In some embodiments, needle cap remover 100 has an elongated cover body 120 configured for at least partially covering a needle cap. Elongated body 120 in some embodiments is sized and shaped to envelope a needle cap, optionally tightly. Elongated body 120 optionally includes a bore 124. For example, body 120 and/or bore 124 may be cylindrical. Alternatively, a portion of body 120 and/or bore 124 may be conical for example being tapered and/or having the shape of a conical section and/or funnel shaped. Body 120 has a distal end B which in some embodiments is found in proximity to a top portion of a needle cap, i.e. the portion having the needle receiving bore. Body 120 has a proximal end A, which in some embodiments is found in proximity to a bottom portion of a needle cap, i.e. the portion closest to the needle tip.

In some embodiments, elongated body 120 comprises at least two bifurcations, optionally dividing body 120 into at least two portions, potentially portion 130 and/or portion 150. In some embodiments, at least a first 130 of the at least two portions has elastic properties, optionally, the two portions are in the form of longitudinal arms. Alternatively or additionally, at least a second 150 of the at least two portions has rigid properties, e.g. is more rigid than first 130 portion. In some embodiments, elastic portions 130 are elastic enough to deflect away from a central axis of the cover body by an angle having a range of 1°-5°. Alternatively, elasticity is enough to enable deflection by an angle having a range of 10°-15°. Alternatively, elasticity is enough to enable deflection by an angle having a range of 15°-20°. In some embodiments, reaching an angle greater than the elasticity range results in plastic deformation of the arms, optionally, reducing the resilient snapping of the arms to their original position. Alternatively, deflection beyond the higher threshold of the range results in breakage of the arms.

In some embodiments, elasticity and/or rigidity is affected by the size, shape and/or position of slits 101 created by the bifurcations, optionally their longitudinal length and/or position along the longitudinal axis of body 120. Alternatively or additionally, the width of slits 101 influences the elasticity of its adjacent portions. An optional range for the longitudinal length of slits 101 is between about 5 mm and about 20 mm. Alternatively, a length of slits 101 is between 7 mm and 10 mm. An optional range for the width of slits 101 is between 0.3 mm and 3 mm. Optionally, the width of slits 101 is 2 mm.

Alternatively or additionally, elasticity and/or rigidity are determined by the position of slits 101 around the perimeter of body 120 and thus according to the perimeter length taken up by elastic portion 130 and/or the perimeter length taken up by rigid portion 150, which are defined by the positions of slits 101. In some embodiments, slits 101 define the partition of the distal portion of body 120 into relatively elastic 130 and relatively rigid 150 portions. Optionally, portions 130 are elastic relative to portions 150, and portions 150 are rigid relative to portions 130. In some embodiments, elastic portions take up to 50% of the perimeter of body 120. Alternatively, elastic portions take up to 40% of the perimeter of body 120. Alternatively, elastic portions take up to 30% of the perimeter of body 120. Alternatively, elastic portions take up to 20% of the perimeter of body 120. Alternatively, elastic portions take up to 10% of the perimeter of body 120.

In some embodiments, at least one intermediate slit 104 is provided, optionally in clastic portion 130, potentially increasing its elasticity. Optionally, intermediate slit 104 is surrounded by body 120 and does not continue all the way to the edge of 120. In some embodiments, intermediate slit 104 is positioned centrally between two of slits 101. Alternatively or additionally, intermediate slit 104 is positioned asymmetrically with respect to slits 101, potentially leading to a favorable direction in reaction to force exertion on device 100. Alternatively or additionally, intermediate slit 104 is positioned in proximity to only one of slits 101. Optionally, intermediate slit 104 has identical dimensions to slits 101. Alternatively, intermediate slit has smaller or larger length. Alternatively or additionally, intermediate slit has smaller or larger width.

In some embodiments, elastic portion 130 and rigid portion 150 have distinct lengths. Optionally, elastic portion 130 extends beyond rigid portion 150 towards the distal end of device 100. In some embodiments, at least some of the elastic portions comprise snaps 132 at their distal end. Snaps 132 are configured in some embodiments to snap body 120 onto the needle cap's top portion. Optionally, snaps 132 create a perimeter which is smaller than the perimeter of the needle cap. In some embodiments, due to the elasticity of elastic portions 130, when device 100 is pushed onto the needle cap, elastic portions 130 are directed away from the central axis of body 120. Optionally, once snaps 132 pass the top portion of a needle cap, they snap into their original position, essentially clasping the needle cover 100 over the needle cap and/or clasping over the top of the needle cover and/or over a top sill of the needle cover. Optionally snaps 132 are angled inward and/or wedged into the top and/or a top sill of the needle cap, being tilted with respect to the central longitudinal axis of the needle cap. In some embodiments, the snaps are deflected towards the central longitudinal axis of the needle cap. Optionally the angle of snaps 132 pulls the distal portion of elastic portion 130 inward (e.g. towards the central axis of the cap) when the cap remover is used to pull the cap.

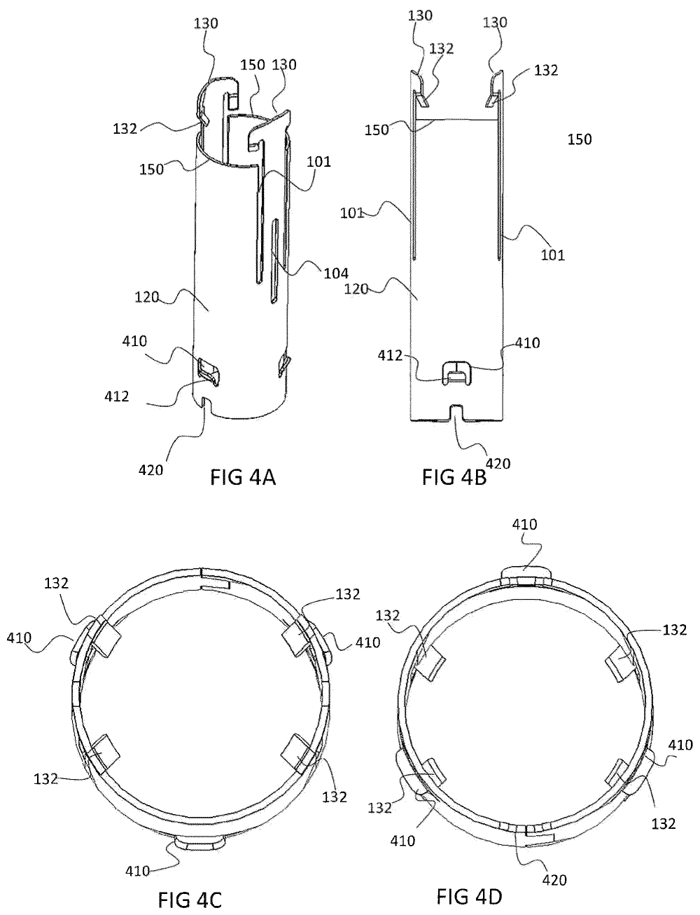

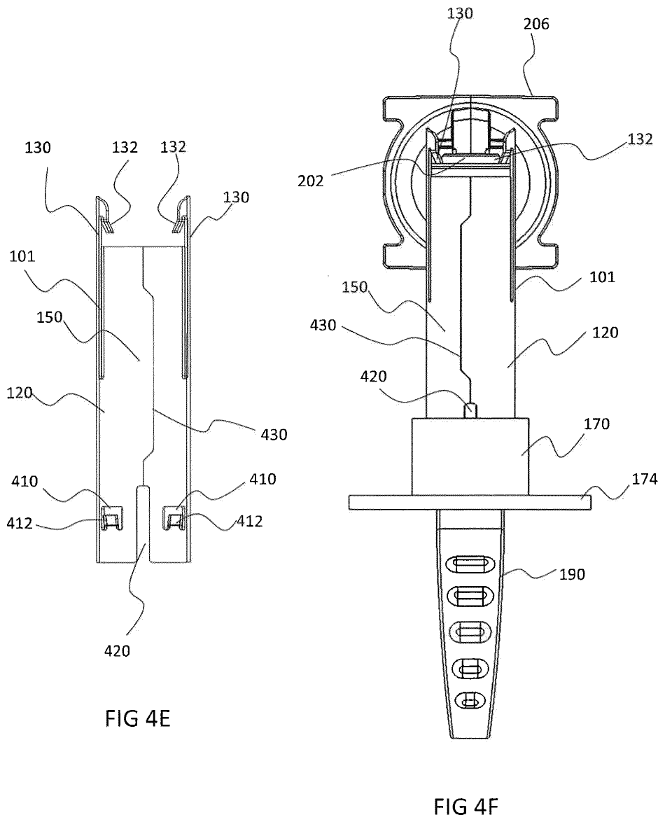

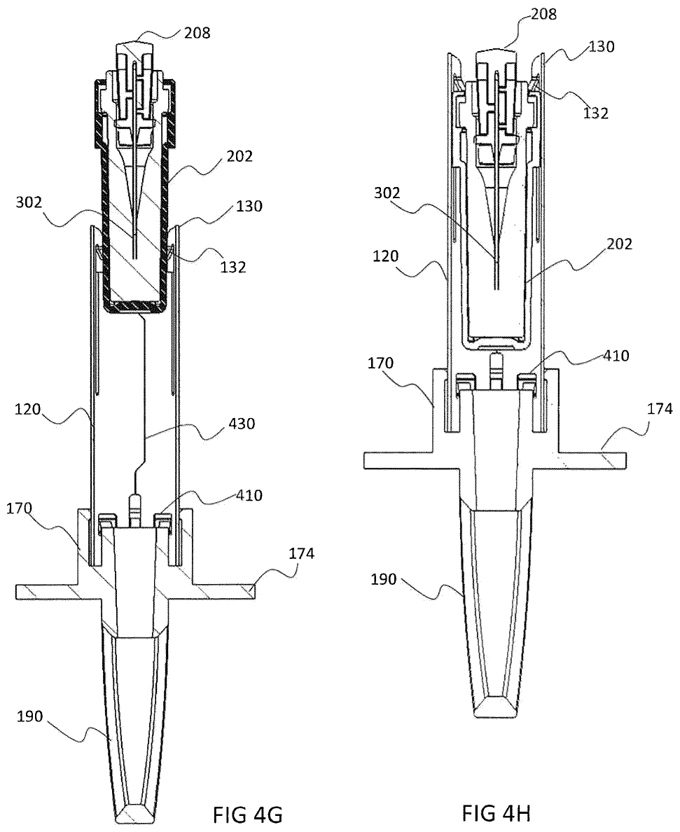

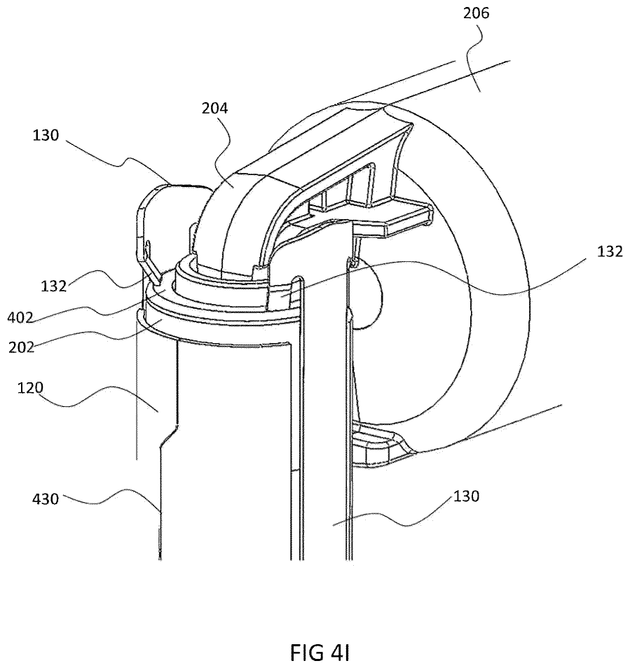

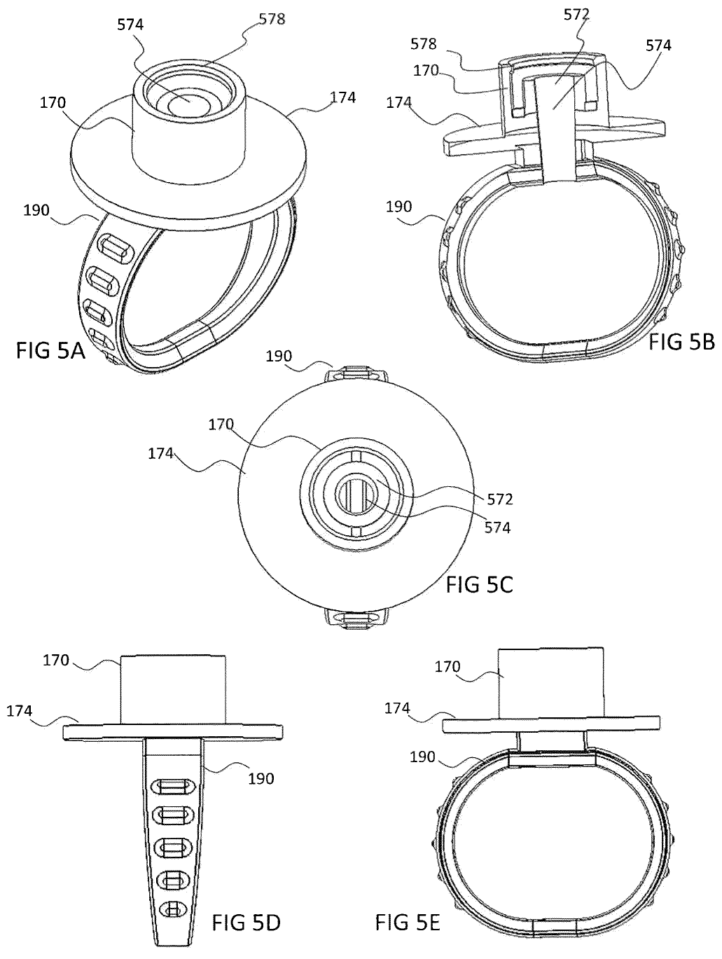

In some embodiments, needle cover 100 further comprises a user handle 190, optionally in the form of a ring as illustrated in FIG. 1. Optionally, user handle 190 is interconnected to cover body 120 by a connector, optionally having connector body 170 for connecting to cover 120 and connector base 174 potentially enabling easier gripping of cap remover 100. In some embodiments, connector body is a separate feature which is connected to body 120, optionally through connector ring 172, further optionally including fastening means 410 configured to interlock ring 172 and body 120, for example as shown in FIGS. 4 and 5. Alternatively, connector base 170 is provided as a continuation of body 120, without being a separate feature. Optionally, fasteners 410 are configured to interlock the body cover with the connector such that a lateral movement of the cover body 120 with respect to its enveloped needle cap is inhibited.

In some embodiments, cover body 120 comprises a recess 420, optionally configured to complement protruding longitudinal guides in the needle cap, thereby guiding the direction of inserting the cover body over the needle cap. In some embodiments, at least 2 recesses 420 are provided. Alternatively, at least 3 recesses are provided. In some embodiments, recesses are provided in the proximal portion of the cover body.

Optionally, connector base 170 and/or body 120 comprise complementary longitudinal sections, which optionally facilitate orientation of the connector base 170 with respect to body 120 when assembling the two together. Longitudinal sections optionally include grooves. Alternatively or additionally, longitudinal sections include protrusions. In some embodiments, an inner portion of body 120 comprises complementary longitudinal sections configured to complement sections provided in a needle cap, potentially aiding in a correct orientation of body 120 with respect to the needle cap. Also potentially, complementary sections can assist in reducing the force needed to assemble cover 120 onto a needle cap.

### 2 Exemplary Usage of the Needle Cap Remover in an Exemplary System of a Drug Delivery Device

In some embodiments, a needle cap remover is used with a drug delivery injector system. Optionally, the injector is provided to the user after the system has been pre-assembled with a medicament, contained in a cartridge having a needle. Optionally the needle is embedded within a housing of an injector system and/or not visible to the user. It is potentially desirable that the medicament and the needle are preserved in sterility until they are used or just before use. For example it is potentially beneficial, to provide the device to the user while the needle is still protected by the needle cap. Optionally the needle cap is internally embedded within the housing. A needle cap remover and/or a handle as provided in accordance with some embodiments of the invention is assembled onto the needle cap, potentially having an internal portion residing inside the housing, while having an external portion outside the housing and being available for a user to pull.

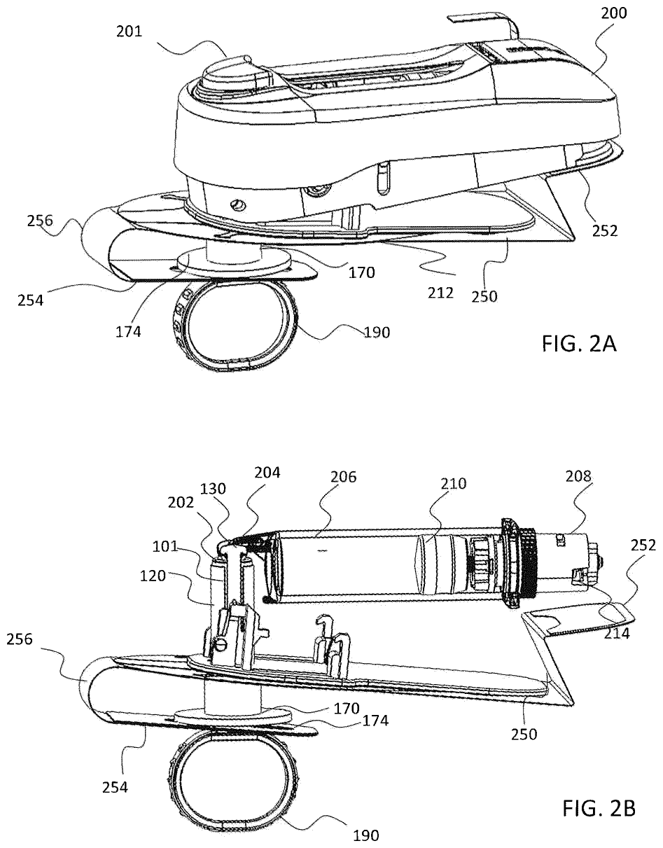

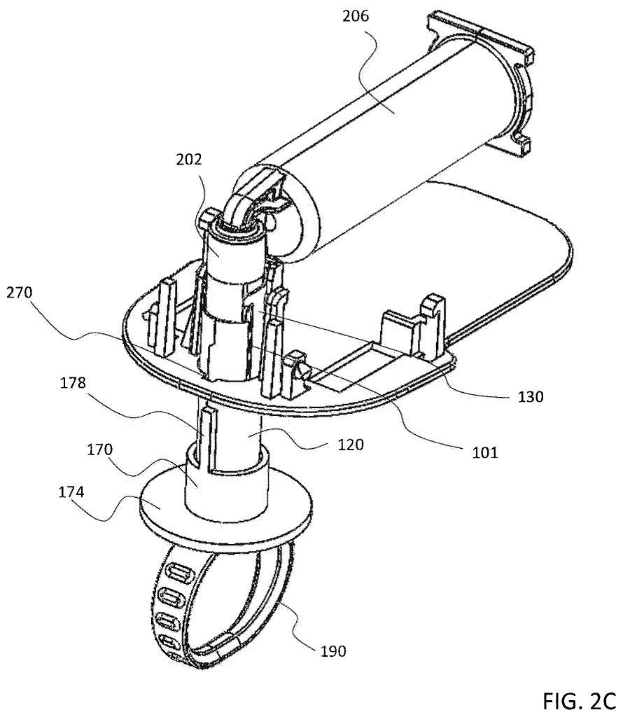

Reference is now made to FIG. 2 illustrating an exemplary drug delivery device in the form of an automatic injector, being in a secure state for example safe for transport, and having cap remover 100 at least partially embedded. In some embodiments, adhesive liner 250 covers the activation zone of the device and/or an adhesive. Optionally, adhesive liner 250 is associated with cap remover 100 such that pulling cap remover 100 also leads to peeling of adhesive liner 250. In some embodiments, cap remover 100 serves as a coupler, optionally synchronizing removal of the needle cap and unsealing of the activation zone of the device, optionally by peeling a protective cover layer.

In some embodiments, an adhesive section of an injector system is provided having two sections, a main adhesive 212 and a secondary adhesive 214, optionally adhesive 214 having a smaller contact area than adhesive 212. A potential advantage of providing at least two adhesive sections 212 and 214 is a guided removal of the device from the user's body, as an adhesive having a smaller contact area is likely to detach first.

In some embodiments, cap remover 100 and/or its associated adhesive liner 250 are associated with a battery isolator. Optionally, once cap remover 100 is pulled and/or adhesive liner 250 is peeled, battery isolator is also removed allowing to power on the injector system. In some embodiments, once power is on, the injector can no longer be turned off and/or repacked in its original state. Alternatively or additionally, once power is turned on, a mobile device is alerted. Optionally, a bar code is provided in the injector system for associating a mobile device to communicate with the injector system.

FIG. 2A illustrates an exemplary injector system having an injector housing 200 and operation button 201, and having cap remover device 100, optionally in proximity to the operation button 201. In accordance with some embodiments, the injector system comprises a two sectional adhesive 214 and 212, optionally covered by adhesive liner 250. In some embodiments, adhesive liner 250 comprises a top portion 252 for covering sections 212 and 214, optionally adhesive 212 and adhesive 250 comprise an aperture for allowing access to the needle and/or needle cap and/or needle cap remover from outside housing 200. In some embodiments, adhesive liner 250 also comprises a bottom portion 254, optionally connected to top portion 252 through bending 256. In some embodiments, bottom portion 254 is associated with needle cap remover 100, optionally by having an aperture for allowing device 100 to extend from within housing 200, through top portion 252 and through bottom portion 254. Optionally, bottom portion 254 is positioned between handle 190 and connector base 174. In some embodiments, connector base 174 comprises a plate, optionally having a diameter larger than bore 124 of body 120. Potentially, once handle 190 is pulled, connector base 174 pulls adhesive liner bottom portion 254, which through bend 256 leads to peeling top cover 252 starting from the device extreme end being closest to bend 256. In some embodiments, connector 170 comprises an orientation feature, such as for example beam 178, optionally sized and shaped to fit into housing 200, for example at slit 270, as shown in FIG. 2C. Potentially, orientation feature 178 is configured to guide the insertion of cap remover device in a specific rotational orientation. Optionally, at least one fastener 350 is provided in connector 170 and/or cover body 120, potentially setting the orientation of connector 170 with body 120.

FIG. 2B illustrates an embodiment of an injector system having a needle cap 202 associated with a cannula 204 of a medicament cartridge 206, resting on a frame of housing 200 of the injector, optionally the injector system is operated automatically by means of motor 208, potentially pushing plunger 210. Putting pressure on the proximal end of the injector (which is optionally covered by front cover 250) optionally pushes needle protector 214 against the frame. Optionally putting pressure on the proximal end of the injector will not activate the injector and/or will not un-shield the needle. Needle cap 202 optionally acts at a physical shield covering the tip of a needle associated with cannula 204.

Optionally, needle cap 202 includes a rigid outer shell and a sterile rubber core. When needle cover 202 is installed over a sterilized needle, the rubber core may protect the sterility of the needle and/or the shell may protect the needle from causing a stick hazard. Alternatively or additionally, a needle is covered with a single cover (either rubber or rigid) that preserves sterility and/or prevents a stick hazard.

For example, a prefilled syringe may be installed into an autoinjector with a needle already covered with needle cover 202. Before shipping the injector, adhesive liner 250 may be installed onto the injector. In some embodiments, a needle cap remover 100 is pushed over cap 202, optionally additionally serving as a needle cap protector. Cap remover 100 in some embodiments includes clasps (for example snaps 132) which engage cap 202. When needle cap remover 100 is removed from the injector, clasps 132 may pull off needle cap 202.

### 3 A Needle Cap Remover Including an Adhesive Liner

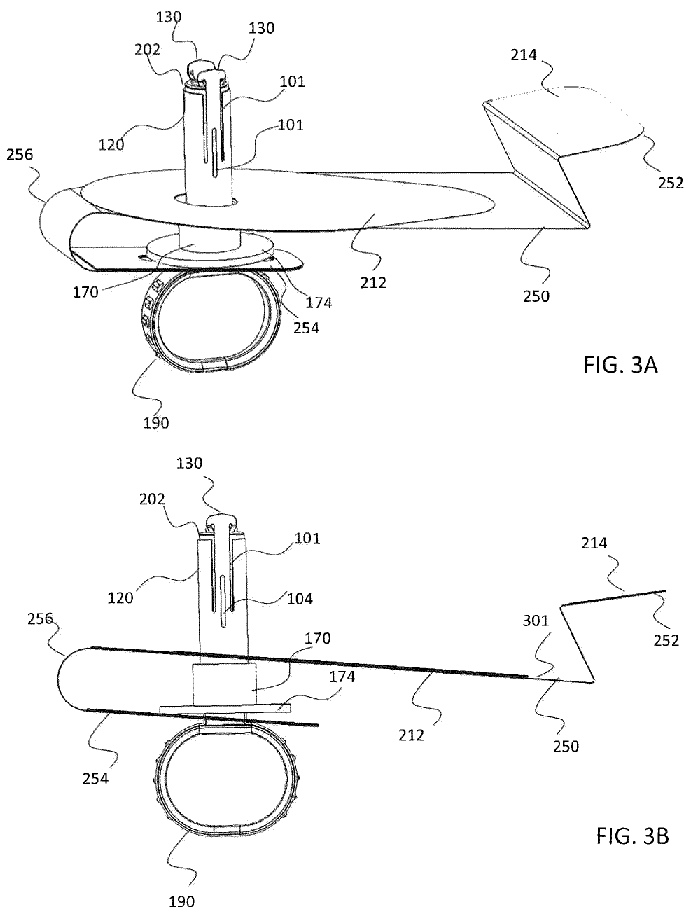

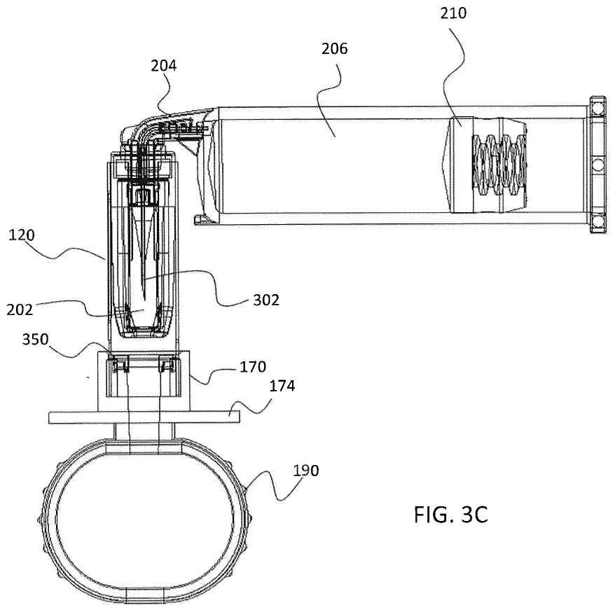

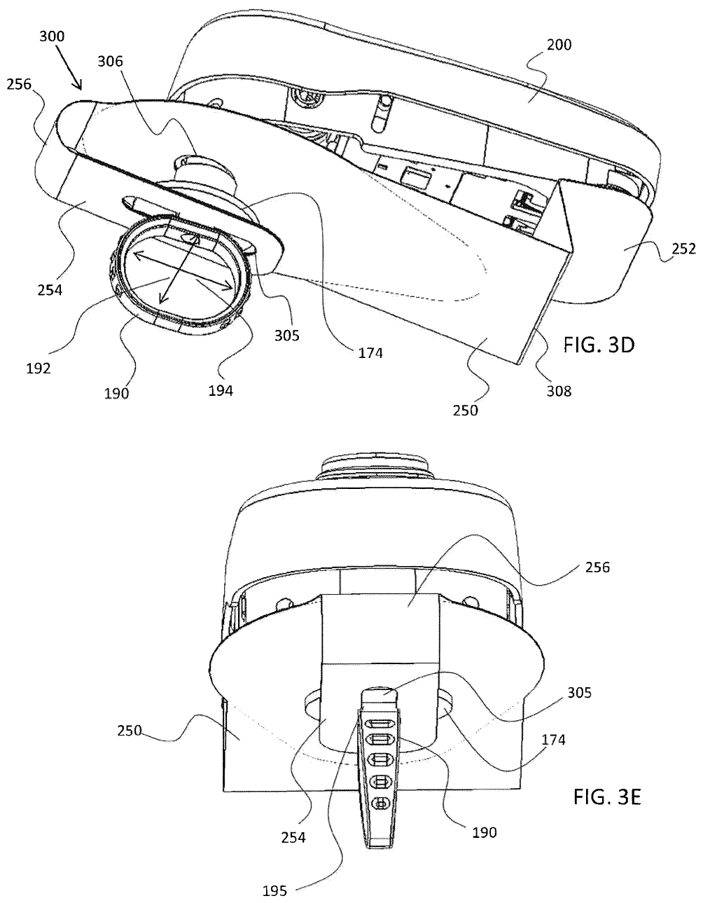

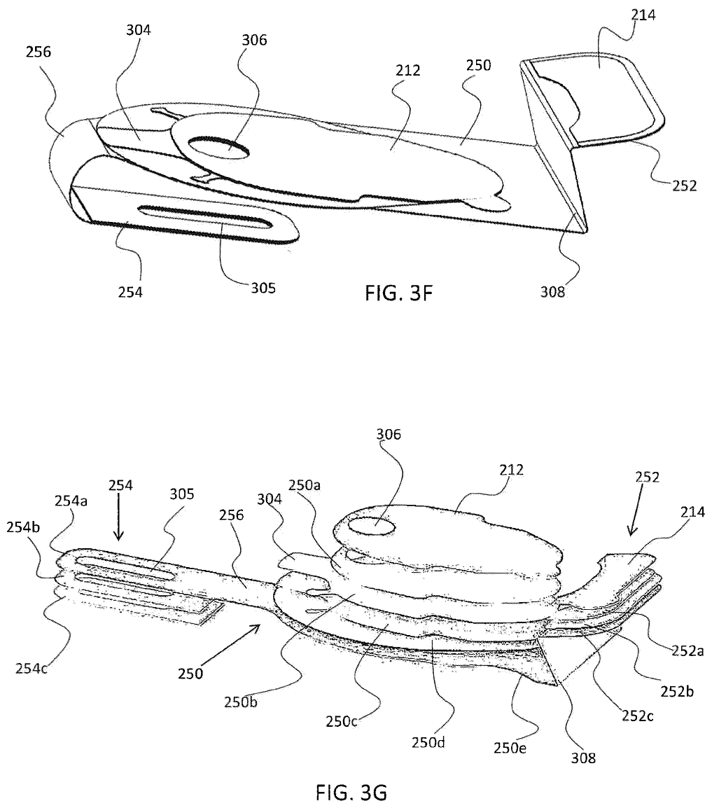

Reference is now made to FIG. 3, illustrating perspective and side views of a needle cap remover optionally having a cover 120, and/or adhesive liner 250 and/or handle 190.

FIGS. 3A and 3B illustrate an injector needle cap remover and adhesive liner in accordance with an exemplary embodiment of the present invention. The cap remover includes for example a handle 190 and/or a needle cap cover 120 and/or an adhesive liner 250 for covering an adhesive 212/214 and/or for covering an activation zone of the injector system. Handle 190 and/or cover 120 and/or cover 250 are optionally connected to a battery insulator. Removing the cover optionally places the injector into an enabled state.

In some embodiments needle cap remover 100 serves as a peeler for an adhesive liner 250. For example, cap remover 100 is optionally hinged and/or flexible. Optionally, handle 190 is located at the center of cap remover 100. In some embodiments, handle 190 does not pull an adhesive liner 250 away from the whole surface of the adhesive 214/212 all at once (an act that would potentially require a large force to overcome the sticking force over a large surface). When handle 190 is pulled, the center portion of cap remover 100 optionally moves away together with adhesive liner bottom portion 254. For example cover 254 is optionally is pulled away in such a way as to peel adhesive liner bend 256 following by peeling of top portion 254 bit by bit. Peeling is optionally from an extreme edge of main adhesive 212 and/or optionally peeled towards the opposite end, finally removing top portion 252 from secondary adhesive 214.

In some embodiments, adhesive 212 is provided onto a hinged plate 301. Optionally, once needle cap remover 100 and cover 250 are removed, a user can attach the device onto an injection site and optionally collapse hinged plate 301 to be parallel to the device. In some embodiments, a sensor senses the orientation of the hinged plate 301 with respect to the device, only allowing operation when the two are aligned. Alternatively or additionally, once plate 301 is parallel, a mechanical mechanism allows for operating the injection, such as for example, by tilting a blocking feature, such as an internal plate.

Reference is now made to FIG. 3C, illustrating a cross section of a side view of a cap remover device assembled onto a needle cap shielding a needle of an injector system, in accordance with some embodiments of the invention. In some embodiments, cover body 120 is configured to be pushed over needle cap 202 which shields a sterility state of needle 302. Optionally, pushing body 120 over cap 202 does not disturb the sterility state of the needle. In some embodiments, cover body 120 comprises a tubular shape, optionally conical. Alternatively, the tubular shape is cylindrical. Alternatively, only a portion of cover body 120 is defined as tubular, and this portion potentially couples at least two arms provided by the cover body 120. Alternatively, cover body is bifurcated to define at least two arms, optionally elastic.

In some embodiments, the elastic portions of cover body 120 allow it to be pushed over cap 202 in spite of having elements defining a smaller perimeter than the largest perimeter of cap 202.

Reference is now made to FIGS. 3D-3G, illustrating exemplary embodiments of a device cover, optionally protecting adhesive properties which are possibly used to couple the device base to a patient's body.

In some embodiments, housing 200 includes in its base a device cover 300. In some embodiments, cover 300 comprises at least two layers, one being an adhesive layer and the other being a liner for protecting the adhesive surface of the adhesive layer. In some embodiments, an adhesive surface of cover 300 assist a user to hold an injector steady on the skin of a patient for an extended period. Optionally, the adhesive surface is intermittent throughout the surface of cover 300. In some embodiments, cover 300 is partitioned at point 308, dividing the adhesive surfaces of the device between main section 250 and secondary section 252, optionally section 252 having a smaller surface area, potentially leading a user in the direction the device is removed, due to the case of extracting the smaller adhered area with respect to the larger adhered area. In some embodiments, folding consists of bending and/or arching, and/or curving and/or winding and/or twisting. Optionally, section 250 and/or section 254 have circular shapes. Alternatively or additionally they may have a shape other than circular, for example oval, rectangular and/or irregular.

In some embodiments, cover 300 comprises at least one portion for connecting to a base of housing 200. In some embodiments, cover 300 comprises an extension 254 extending beyond a surface area of the housing base. Optionally, extension 254 is folded over at section 256 to be positioned to face portion 250 of cover 300.

In some embodiments, cover 300 comprises at least two apertures 305 and 306. In some embodiments, a first aperture 306 comprises in section 250 is sized and shaped to allow insertion of needle cap remover body 120 through it and into the inner portion of the injector device, which comprises the needle. Optionally, aperture 306 extends through the adhesive liner and the protective liner, and any other layer comprised the protective cover 300. In some embodiments, aperture 306 comprises a circumference shape matching a circumference shape of the needle cap remover body 120 and optionally, having a width being no more than about 1 mm, or about 0.7 mm, or about 0.5 mm, or about 0.3 mm wider than the width of the remover body 120.

Optionally, the adhesive liner is folded over at point 256, forming an extension 254 which directs a linear unsealing force into a guided peeling force starting from the folded edge. In some embodiments, a second aperture is provided in extension 254 of cover 300 which extends from the cover portions being connected to the device base, such as section 250 and/or 252, through bent portion 256. Optionally, the second aperture 305 is used to assemble section 254 onto the connector of needle cap remover body 120. In some embodiments, needle cap remover device serves as a coupler, coupling the needle cap at its distal side and the user handle 190 at its proximal side, optionally through connector 170. In some embodiments, the user handle comprises a protruding element opposite said coupler, e.g. being proximal to the proximal end of the needle cap remover 100.

In some embodiments, second aperture 305 comprises an elongated orifice. In some embodiments, the elongated orifice is sized to be pulled over a user handle 190 extending from needle cap remover body 120, optionally connected to it through connector 170. Optionally, user handle 190 comprises a protruding element having a long dimension 194 and a short dimension 192. In some embodiments, elongated orifice 305 has a length which is longer than short dimension 192 and longer than long dimension 194. In some embodiments, user handle 190 has a widening portion towards its connection with the connector 170, optionally ending in shoulder 195, shown in FIG. 3E. In some embodiments, a width of orifice 305 is shorter than a width defined by shoulder 195. In some embodiments, orifice 305 is at least partially made of a material which is clastic and/or resilient enough to allow pushing the orifice over the shoulder, despite its shorter width. Optionally, extension 254 is held over handle 190 by overhanging shoulder 195. Alternatively or additionally, shoulders 195 include the widest portion of the narrow portion of handle 190, sized to be not smaller than a width of orifice 305. Optionally, orifice 305 is stabilized onto shoulders by friction.

In some embodiments, an additional supporting layer is engaged with the protective cover, optionally being intermittent and not continuous over an area of cover 300. In some embodiments, supporting layer is made of a material being rigid enough to increase a rigidity of the protective liner, potentially providing mechanical strength for cover 300. Optionally, supporting layer is provided at least in the sections comprising apertures 305 and 306. In some embodiments, supporting layer is not found in extension 256.

In some embodiments, an injector housing is provided with cover 300 being at least partially connected to its base, optionally by an adhesive layer. Optionally, the injector is assembled by interlocking a cartridge having a capped needle within an inner portion of housing 200, such that the cartridge central axis is perpendicular to the cover, and the capped needle is centrally aligned with aperture 306. In some embodiments, a distal end of needle cap remover is inserted into the inner portion of the base of housing 200 through aperture 306, and is pushed over the capped needle. Optionally, the proximal end of cover body 120 remains protruding externally to housing 200.

In some embodiments, a user handle is connected to the proximal portion of body 120, optionally through connector 170. In some embodiments, user handle 190 is characterized by a protruding element extending outwardly from the base and having a short dimension 194 and a long dimension 192. In some embodiments, extension 254 is folded over the user handle 190 by first orienting aperture 305 to face the shorter dimension 194 of handle 190, followed by rotating the cover extension 254 over the handle until the aperture 305 is oriented to face the longer dimension 192.

In some embodiments, aperture 306 and 305 are centrally aligned. Alternatively or additionally, aperture 305 slides across along the long dimension 192 of handle 190, such that a longitudinal axis is not centrally aligned with aperture 306 and/or with handle 190. Potentially, sliding aperture 305 with respect to handle 190 further stabilizes its position and prevents rotation of the aperture 305 in the reverse direction. Optionally, slide of aperture 305 with respect to handle 190 is produced with extension 256 is stretched between section 250 and extension 254.

In some embodiments, handle 190 comprises a widening portion extending in a direction from a proximal end defined as being away from housing 200 and widening when approaching a distal end of the handle defined as being proximal to housing 200, optionally being widest as shoulder portion 195. In some embodiments, aperture 305 is passed over the widening portion and is then constrained against the widened portion, potentially being held in place by friction forces. Alternatively, aperture 305 is passed over the widening portion a narrower portion being distal to the shoulder 195, and potentially the extension 254 is stabilized in place by overhanging an edge of aperture 305 over the widening portion, and/or shoulder 195.

In some embodiments, needle cap remover 100 serves as a peeler for cover 300, optionally at least its protective liner layer. For example, needle cap remover 100 is engaged with extension 254 through orifice 305. Optionally, user handle 190 which is optionally connected needle cap remover body 120, does not pull cover 300 directly away from the whole surface of the device base all at once (an act that might require a large force to overcome the sticking force over a large surface). In some embodiments, when handle 190 at is pulled, extension 254 pulls with it section 256 followed by section 250. In some embodiments, extension 304 guides the peeling initiation direction of section 250. In some embodiments, folded section 256 may unfurl, unfold, stretch and/or bend to allow a certain distance to build up. Optionally, unfolding of section 256 together with peeling of extension 304, converts the linear force away from housing 200 to a peeling force at the edge of adhesion of adhesive cover adhesive cover. The peeling force may optionally be along the surface of base of housing 200 and/or the peeling force may be directed away from the surface at an angle. For example, the angle of the peeling may range for example between 60-90 degrees and/or between 30-60 degrees and/or between 0 and 30 degrees.

FIG. 3G illustrates details of possible structure of cover 300, having at least two active zones including an adhesive formed on a surface according to some embodiments of the current invention. In some embodiments a wall of a device (for example base of housing 200) may have an active surface (for example adhesive 250a and/or 250d). Optionally the adhesive may be formed in layers. For example, as illustrated in FIG. 3G, adhesive portion of cover 300 includes three layers. A first layer 250a includes a dual sided adhesive. Adhesive may for example, join a semi-stiff membrane 250b to the base 212. Membrane 250b may extend beyond the edges of the base, optionally extending in extension 304. Potentially, a semi-stiff extension beyond the edges of the base may in some embodiments make the injector adhere more strongly the skin of a user. In some embodiments, supporting membrane 250c is provided.

In some embodiments, the adhesive cover removal force ranges between 10-150 gr/cm. The adhesive cover removal force may be lower than the bending inertia of the adhesive membrane 250a. For example this may prevent bending of membrane 250b when removing the cover. An external face of membrane may optionally include an active surface. An active surface for the purpose of this application may be a surface that has active modality wherein the surface interacts with an external element facilitating the functioning of the device and an inactive modality wherein it substantially does not interact with the external element. For example, prior to enablement of the injector adhesive 250d may be inactive and/or protected by adhesive cover 250e.

Optionally, sections 250, 254 and/or 252 have different numbers of layers. In some embodiments, section 254 comprises adhesive layer 252a connecting base portion 214 to liner 252c and its supporting layer 252b. In some embodiments, extension 254 comprises extension of section 250 in the form of layer 254a, and further comprises adhesive layer 254b connecting to supporting layer 254c.

### 4 Exemplary Bifurcated Needle Cap Remover

Reference is now made to FIG. 4 illustrating various views of needle cap cover body 120, in accordance with some embodiments of the invention. In some embodiments, cover body 120 is provided as a cylinder having a bore 124. In some embodiments, a distal portion of body 120 is bifurcated. Optionally, at least two slits 101 provide the bifurcated portions, optionally creating at least two distinct portions.

In some embodiments, at least 4 slits 101 are provided such that at least 4 distinct portions are available at the distal end of body 120. Optionally, at least two of the portions comprise clastic portions 130. Alternatively or additionally, at least two of the portions comprise rigid portions 150. Optionally, at least two elastic portions 130 are positioned substantially diametrically to one another and/or optionally at least two rigid portions 150 are positioned substantially diametrically to one another.

In some embodiments, clastic portions 130 have a length extending beyond a top sill of a needle cap, for example, beyond 0.5 mm, 1 mm, or beyond 2 mm, or beyond 3 mm, or any length smaller, larger or intermediate. Optionally, elastic portions 130 extend in a range having length of about 3 mm to about 10 mm beyond the top rim of bore 124. In some embodiments, extended elastic portions comprise snaps 132, configured to overhang a top sill of needle cap 202. In some embodiments, snaps 132 comprise projections, optionally extending into an inner radial direction of bore 124. Optionally, the projections have a length of about 0.2 mm. Alternatively, the projections have a length of about 0.1 mm. The projections have a length of about 0.3 mm, or any length smaller, larger or intermediate to the mentioned lengths herein.

In some embodiments, intermediate slits 104, optionally not extending all the way to the distal rim of bore 124, are provided, optionally in elastic portions 130. Potentially, a configuration of positioning slits 101 and/or 104, and/or their size and/or shapes affect an clastic extent of elastic portions 130 and a rigidness extent of rigid portions 150. In some embodiments, the configuration and/or shape and/or size of slits 101 and/or 104 are configured to allow a small amount of force to push body 120 onto the needle cap and a large amount of force to pull body 120 away from the needle cap, optionally resulting in removal of the needle cap together with pulling of body 120. For example, a small amount of force is in the range of about 50 g to about 400 g. Alternatively, small force is in the range of about 100 g to about 300 g. Alternatively, small force is in the range of about 100 g to about 150 g. Optionally, a large amount of force is in the range of about 0.5-1.5 Kg. Alternatively, large amount of force is in the range of about 0.75 Kg to about 1.25 Kg. Alternatively, large amount of force is no more than 1 Kg.

In some embodiments, body 120 comprises at least one fastener 410, optionally having fastener lock 412 such as for example a projection, optionally located at the proximal portion of body 120. Potentially, fastener 410 is configured to snap and/or lock with complementary portions in connector body 170, potentially stabilizing body 120 in place. Alternatively or additionally, connector body 170 connects body 120 with handle 190. A potential advantage of providing multiple parts is to enable more degrees of freedom in an assembly process. In some embodiments, at least 3 fasteners 410 are provided, optionally equidistant from each other.

Reference is now made to FIGS. 4E and 4F, illustrating another embodiment of a needle cap remover, in accordance with some embodiments of the invention, and having at least four fasteners 410. FIG. 4E illustrates a front view of body cover 120 and FIG. 4F illustrates a front view of body 120 when connected to connector body 170 and while enveloping cap 202, which shields a needle connected to cannula 204 which is coupled to injector 206. Fasteners 410 are optionally coupled in their position to the position of the bifurcations. In some embodiments, fasteners 410 are provided in the rigid portions 150 of cover body 120. Optionally, cover body 120 comprises crack 430, possibly in the form of a non-straight line. A potential advantage of crack 430 is allowing more flexibility. A potential advantage of a non-straight form is allowing more stability.

Reference is now made to FIGS. 4G and 4H, illustrating a cross section front view of the cap remover being pushed over the needle cap, in accordance with some embodiments of the invention, wherein FIG. 4G illustrates the needle cap remover being pushed mid-way along the needle cap and FIG. 4H illustrates the needle cap remover after snapping into place and having snaps 132 overhanging the top sill of the needle cap.

In some embodiments, the perimeter of the cap is not uniform, and it optionally includes a larger perimeter at its top end, i.e. the location where the needle projects outward from the syringe body. In some embodiments, hooks 132 are configured to define a perimeter which in its relaxed, unstressed state, can contains the perimeter of the bottom portion of cap 202, but cannot contain the perimeter of the top portion of cap 202. In some embodiments, the top portion of cap 202 comprises depressions for clasping hooks 132.

Reference is now made to FIG. 4I, illustrating a perspective partial view of cartridge 206, having a needle cap 202 protecting a needle connected to cannula 204, assembled with a cap remover device in accordance with some embodiments of the invention. In some embodiments, hooks 132 are configured to deflect elastic portions 130 away from the central axis of needle cap 202, optionally when passing by perimeters which are larger than defined by hooks 132. In some embodiments, hooks 132 are configured to overhang a top sill 402 of cap 202, optionally once the hooks pass the top portion of cap 202.

In some embodiments, hooks 132 include projections in a direction towards a central axis of the remover device. Optionally, the projections 132 are perpendicular to the longitudinal axis of the cover body 120. Alternatively, the projections are tilted towards the proximal portion of cover body 120. In some embodiments, the tilting of the projections 132 affects their resistance when pulling the cover body 120 with respect to cap 202. Optionally, a pulling force over cover body 120 causes projections 132 to increase their tilting angle with respect to the longitudinal axis of cover 120, potentially causing a mechanical interference resisting the pulling of cover body 120 without also pulling needle cap 202.

### 5 An Exemplary Connector for a Needle Cap Remover

Reference is now made to FIG. 5, illustrating a connector in the form of connector 170, in accordance with some embodiments of the current invention. In some embodiments, connector body 170 comprises a bore 574 sized and shaped to fit a needle cap remover cover body such as cover body 120, and/or to fit the needle cap proximal portion. In some embodiments, connector body 170 is provided with at least one complementary portion configured to lock connector body 170 onto cover body 120, optionally through fastener 410. In some embodiments, a connector undercut 578 is provided, enveloping at least a portion of connector bore 574 and optionally configured to clasp onto at least one fastener 410. In some embodiments, fastener 410 is shaped to allow easy insertion of connector body 170 in the direction towards the distal portion of cover body 120, but optionally clamp the connector 170 to the cover body 120 once undercut 578 has passed the fasteners position. In some embodiments, fasteners 410 are configured to prevent a lateral movement of the needle cap remover cover body with respect to the needle cap.

In some embodiments, handle 190 is connected to connector body 170, optionally through connector 572. Alternatively or additionally, handle 190 is made as a single unit together with connector body 170 and/or connector base 174.

### 6 Assembling a Needle Cap Remover Onto a Needle Cap

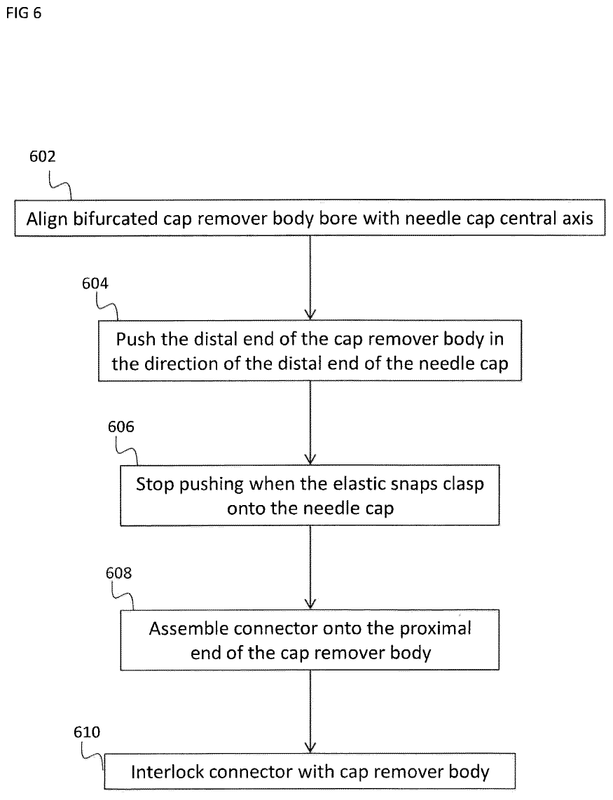

Reference is now made to FIG. 6, having a flow chart illustrating an exemplary assembly of needle cap remover onto a needle cap, in accordance with some embodiments of the invention. In some embodiments, a cap remover body is aligned with a needle cap central axis 602. Alignment potentially prevents an excessive force exertion when pushing the remover onto the cap, which could lead to harming the needle, and/or disturbing the sterility of the inner zone of the needle cap.

In some embodiments, once the cap remover is substantially aligned with the needle cap, the cap remover is pushed towards the needle cap 604 optionally by inserting the proximal end of the needle cap into the distal end of the cap remover and pushing the distal end of the cap remover towards the direction of the distal end of the needle cap. Having bifurcations in the cap remover body potentially leads to elastic portions in the cap remover. In some embodiments, once the cover is pushed, the elastic portions, comprising snaps which optionally define a smaller circumference than the circumference of the needle cap, are pushed away from the central longitudinal axis of the cap remover.

In some embodiments, pushing of the cap remover stops when the elastic snaps reach beyond the top portion of the needle cap 606. In some embodiments, an interlocking mechanism prevents pushing of the cap remover beyond a certain point above the needle cap, such as for example, fasteners 410. Alternatively or additionally, the pushing of the cap remover stops when a sensor senses a reduction in the force resisting the push. In some embodiments, the snaps bounce into place thanks to the elastic properties of the elastic portions, optionally leading to the snaps overhanging a top sill of the needle cap.

Optionally, a connector, for example in the form of a clamp, is assembled onto the proximal end of the cap remover body 608. The connector potentially holds the cap remover body still with respect to the housing of an injector, and/or allow connection of the cap remover to additional features, such as a user handle and/or a liner covering operation features, for example adhesive portions and/or battery insulators.

In some embodiments, connector is pushed onto remover body until it interlocks in place 610, optionally by interlocking with at least one fastener 410 provided in the cap remover body.

Alternatively or additionally, the clamp, optionally already associated with a user handle, is interconnected to cap remover body prior to its enveloping onto the needle cap.

### 7 Assembling a Needle Cap Remover Having an Adhesive Liner, Onto a Needle Cap

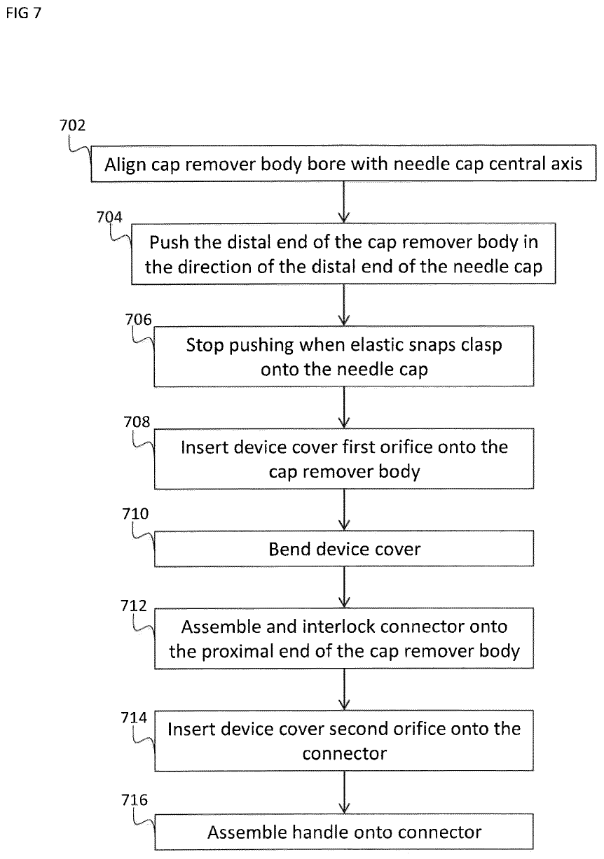

Reference is now made to FIG. 7, showing a flow chart illustrating an exemplary assembly process of a needle cap remover associated with a device cover, such as for example an adhesive liner, in accordance with some embodiments of the invention.

In some embodiments, aligning the central axis of the cap remover body with central axis of the needle cap 702 is done initially to prevent unnecessary force exertion. In some embodiments, the cap remover is then pushed onto the needle cap 704 as shown in 604, and optionally pushing is stopped when the body is clasped onto the cap 706, optionally by means of elastic snaps.

In some embodiments, the cap remover device is being inserted into a first orifice provided in a device cover 708, optionally comprising an adhesive liner. In some embodiments, a connector is assembled onto the cap remover 712, optionally securing the cover onto the cap remover.

In some embodiments, the device cover is bent 710, optionally at a portion which extends beyond the border of the injection system, and the connector body is inserted into a second orifice provided in the cover 714. Optionally, the second portion of the cover is secured after assembly of the user handle onto the connector 716.

### 8 Exemplary Assembly Process of an Automatic Needle Injector Device

Reference is now made to FIG. 8, illustrating an exemplary process of an assembly process of an automatic needle injector device, in accordance with some embodiments of the invention.

In some embodiments, a base of a needle injector housing is supplied 802, optionally being attached to a protective cover. In some embodiments, the protective cover comprises at least one adhesive layer, optionally for positioning and stabilizing the injector over a user's body. In some embodiments, the adhesive layer is protected by a second layer being a protective liner.

In some embodiments, a prefilled cartridge, optionally having a bend needle path, is positioned inside the base and interlocked to be engaged with it 804. Optionally, the cartridge is positioned such that a central axis of the cartridge to be parallel to the base-cover. In some embodiments, the cartridge comprises a capped bent needle. Optionally, the bent needle is centrally aligned with an orifice provided within the base, potentially allowing access from the outside of the injector housing, to the needle optionally residing within the injector housing. In some embodiments, the device cover covering the base also comprises a first aperture, optionally matching to the orifice of the base.

In some embodiments, a needle cap remover is passed from the outside portion of the base into the inside portion of the base, through the first aperture of the device cover 806. Optionally, the needle cap remover is pushed onto the capped needle. In some embodiments, the first aperture is sized and shaped to fit at least the circumference of the needle cap remover. In some embodiments, the needle cap remover is placed onto the capped needle in its distal end, optionally being in the inner portion of the injector. Optionally, the needle cap remover comprises in its proximal end a user handle, potentially being used to remover the needle cap remover with the needle cap.

In some embodiments, the needle cap remover is placed onto the capped needle while being attached to the user handle. Optionally, a cover edge which is not attached to the outer portion of the base, is folded over and attached to the user handle 808. In some embodiments, when pulling the user handle linearly, the cover edge is pulled with the handle and its connected needle cap remover, while the cover portion attached to the device is peeled from the direction of the edge.

In some embodiments, the user handle comprises a protruding member having a short dimension and a long dimension. Optionally, the cover is attached to the user handle by passing a second aperture of the cover over the protruding portion of the user handle. In some embodiments, the second aperture is assembled onto the user handle by orienting the second aperture of the cover to face the short dimension of the handle and rotating the cover onto the protruding element until the second aperture is oriented to face the long dimension.

### 9 Exemplary Multi-Part Cap Remover

In some embodiments, a cap remover is provided by having multiple parts. Optionally, the parts are assembled to a complete cap remover only while and/or after assembling onto the needle cap.

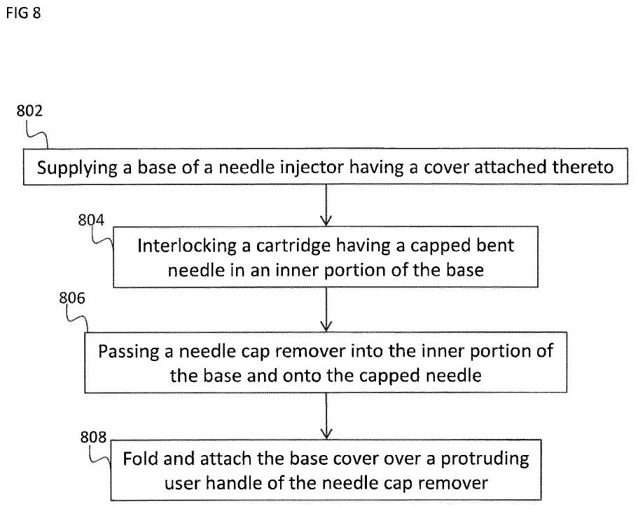

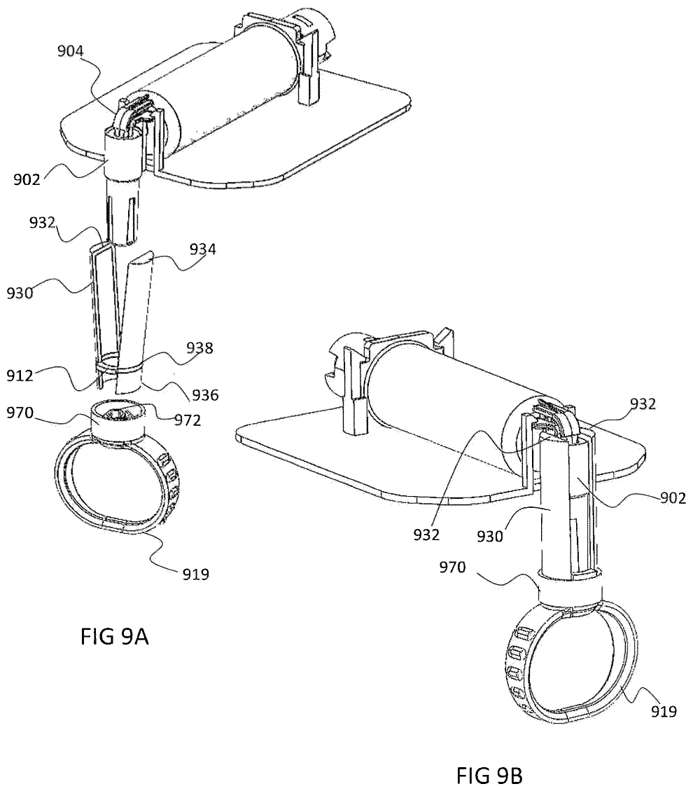

Reference is now made to FIGS. 9A-B, illustrating an exemplary embodiment of a multi part cap remover in accordance with some embodiments of the current invention. In some embodiments, a needle cap remover is provided having at least two elongated arms 930, sized and shaped to fit a longitudinal portion of a needle cap wall 902, optionally serving to protect cannula 904. Optionally, a distal portion 934 of the arms comprise enclosing members 932, optionally sized and shaped to fit a top surface of a needle cap. In some embodiments, a connector body 970 is sized and shaped to enclose over the at least two arms 930, optionally at their proximal portion 936, optionally leading to their clasping over the cap.

In some embodiments, an intermediate portion 938 of arm 930 comprises at least one fastening member; such as for example ring 912. Optionally, member 912 is configured to interlock with fastener 972 provided within connector body 970.

FIG. 9A illustrates an exemplary open configuration of the cap remover, having arms 930 tilted away from the longitudinal axis in their distal portion 934, optionally while being held together by ring 912. FIG. 9B illustrates an exemplary closed configuration, illustrating the enveloping of arms 930 around the needle cap, and their optional securing mechanism provided by connector 970, which optionally further comprises a user handle 919.

### 10 Assembling a Multi-Part Cap Remover Onto a Needle Cap

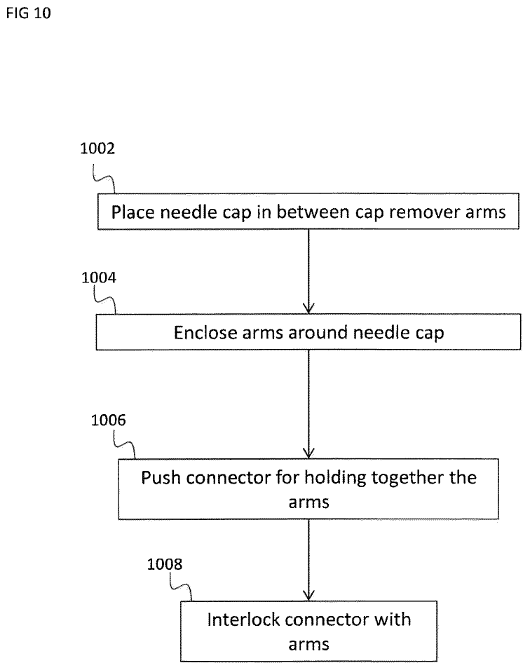

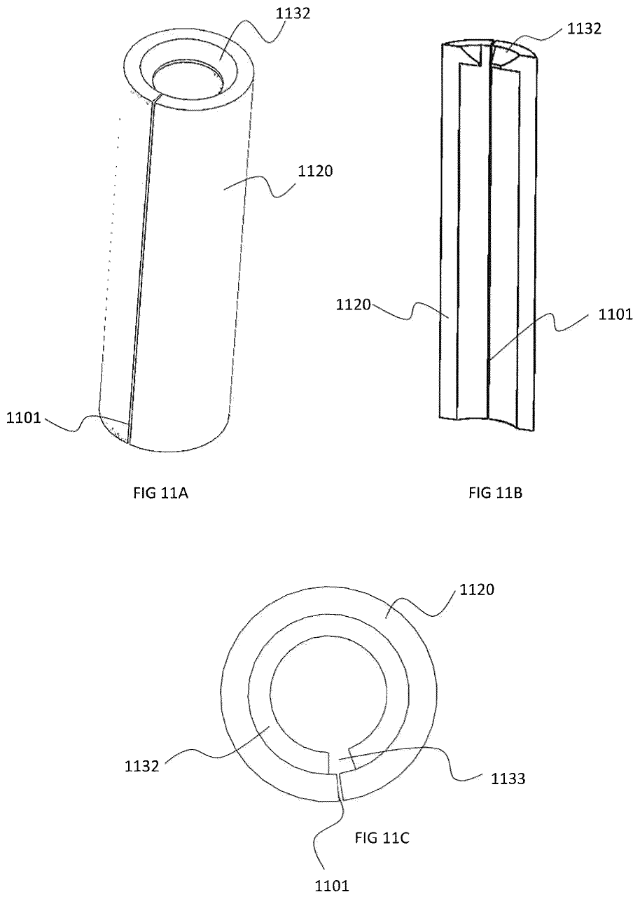

Reference is now made to FIG. 10, showing a flow chart illustrating an exemplary assembly process of a multi-part needle cap remover, in accordance with some embodiments of the current invention.

In some embodiments, a needle cap is placed in between the arms of a multi-part cap remover 1002. A potential advantage of having multiple parts is a flexibility in the perimeter defined by the arms. Optionally when the arms are open, they define a perimeter which is larger than the perimeter of the needle cap, potentially allowing their placement with a relatively low amount of force and/or low precision of aligning the central axis of the cap with the central axis of the cap remover.

Optionally, the arms enclose over the cap 1004 when a securing connector is pushed 1006 onto the proximal end of the arms, tilting them towards the needle cap. In some embodiments, the needle cap remover is held securely over the needle cap once the securing connector interlocks with the arms 1008 leading to securement of the arms over the needle cap. In some embodiments, the connector interlocks with the arms by clasping over the coupling element which mechanically couples the two arms, optionally the coupling element being a ring.

In some embodiments, no more than 25 g of force is exerted in order to interlock the connector with the arms, leading to their closed configuration. Alternatively, no more than 50 g force is exerted. Alternatively, no more than 100 g force is exerted. In some embodiments, in order to pull the needle cap no more than 0.5 Kg force is applied. Alternatively, no more than 0.7 Kg force is applied. Alternatively, no more than 0.9 Kg force is applied. Alternatively, no more than 1 Kg force is applied. Alternatively, no more than 1.2 Kg force is applied. Alternatively, no more than 1.5 Kg force is applied.

### 11 Exemplary of a Needle Cap Remover Having a Single Arm

In some embodiments, a cover body comprising a single arm 1120 is provided. Optionally, arm 1120 comprises a tubular shape. In some embodiments, slit 1101 is provided along the central longitudinal axis of arm 1120, optionally extending along the entire length of arm 1120. Potentially, slit 1101 provides elasticity to arm 1120, enabling the width of 1120 to open when force is exerted. In some embodiments, undercut 1132 is provided in at least a portion of the inner circumference of the top sill of arm 1120. Undercut 1132 is optionally sized and shaped to allow relatively easy insertion against a surface, together with relatively difficult removal, possibly due to its snap enablement. In some embodiments, aperture 1133 is provided to allow room for features comprised in the injector, which may hinder the space available for arm 1120.

As used herein the term “about” refers to ±25%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A system for medicament delivery, comprising:
(a) a cartridge containing a medicament and being in fluid communication with a needle, said needle enveloped by a needle cap;
(b) a housing for containing said cartridge and having an orifice for allowing access to said needle cap and its associated needle; and
(c) a needle cap remover device comprising:
(i) at least two elastic arms coupled to define an unstressed width greater than the outside of a width of a top sill of the needle cap, said elastic arms envelop at least a portion of said needle cap, wherein a proximal end of said elastic arms can be accessed through said orifice;
(ii) a rigid portion, said at least two elastic arms extending beyond said rigid portion towards a distal end of the needle cap remover device; and
(iii) a distal end of each of said at least two elastic arms including at least one snap, said snaps defining an unstressed width smaller than the width of the top sill of the needle cap,
wherein said at least two snaps overhang the top sill of the needle cap, and when said proximal end of said elastic arms is pulled, said snaps cause said needle cap to pull with the needle cap remover device.

2. The system of claim 1, wherein each of said at least two snaps comprise two hooks inflecting towards a central axis of the needle cap and being angled towards the top sill of the needle cap.

3. The system of claim 2, wherein said hooks are symmetrically arranged around a central axis of said top sill of the needle cap.

4. The system of claim 2, wherein said needle cap remover device comprises two of said at least two arms, and wherein each of said two arms comprises two of said hooks.

5. The system of claim 1, further comprising an adhesive layer having a protective liner, said adhesive layer connected to said housing, wherein said protective liner extends beyond a surface of said housing and positioned between said needle cap remover device and a connector.

6. The system of claim 5, wherein, when said needle cap remover is pulled linearly, said protective liner is peeled from said housing.

7. The system of claim 1, wherein pulling said needle cap remover device together with the enveloped needle cap requires a force being no more than 1 kg at most.

8. A method of assembling a needle cap remover onto a needle cap, comprising:
(a) aligning a longitudinal axis of the needle cap remover to be colinear with a longitudinal axis of the needle cap; and
(b) inserting the distal end of the needle cap remover in the direction of a top portion of the needle cap while deflecting at least two elastic elements of said needle cap remover in a direction away from the central axis of the needle cap, said deflecting is provided by a plurality of hooks coupled to a distal end of each of the at least two elastic elements and defining a smaller perimeter than a perimeter of the needle cap, said needle cap remover further including a rigid portion, said at least two elastic elements extending beyond said rigid portion towards the distal end of the needle cap remover,
wherein said inserting is provided until said plurality of hooks extend beyond the top portion of the needle cap and snap inwardly towards the central axis of the needle cap, thereby overhanging the top portion of said needle cap, causing the elastic elements to snap from said deflecting towards the central axis of the needle cap.

9. The method according to claim 8, wherein the needle cap is comprised within an injector system, and said method further comprises inserting the needle cap remover through an orifice in the injector system having access to the needle cap.

10. The method according to claim 8, further comprising interlocking a proximal portion of said needle cap remover with a connector having a user handle.

11. The method according to claim 8, further comprising associating said needle cap remover with a battery insulator in an injector, such that removing the needle cap remover places the injector into an enabled state.
